(19) United States
(12) Patent Application Publication
(10) Pub. No.: US 2026/0284280 A1
(43) Pub. Date: Sep. 24, 2026

(54) VISUAL AND TACTILE VERIFICATION AND SECURING SYSTEM FOR NEEDLE THORACOSTOMY (NT)

(71) Applicant: THE TRUSTEES OF THE UNIVERSITY OF PENNSYLVANIA, Philadelphia, PA (US)

(72) Inventors: Jonathan BAR, Philadelphia, PA (US); Neil RAY, Philadelphia, PA (US); Nicholas BENSON, Ridley Park, PA (US)

(21) Appl. No.: 19/479,514

(22) PCT Filed: May 3, 2024
(86) PCT No.: PCT/US2024/027607

§ 371 Date: Oct. 28, 2025

Related U.S. Application Data
(60) Provisional application No. 63/500,130, filed on May 4, 2023.
(60) Provisional application No. 63/597,012, filed on Nov. 8, 2023.

(51) Int. Cl. A61M 1/04; A61M 25/00; A61M 25/06; A61M 25/10

(52) U.S. Cl. CPC A61M 1/04; A61M 25/0097; A61M 25/10182; A61M 25/0606; A61M 2205/582; A61M 2205/583

(57) ABSTRACT

A device, comprising: a syringe body, the syringe body including (i) a compartment having a fluid disposed therein, (ii) a plunger in fluid communication with the compartment, (iii) a resilient member biased against the plunger, and (iv) a needle in fluid communication with the compartment. When the needle is advanced into a pleural space, the resilient member effects movement of the plunger such that pleural air is communicated to and visible in the fluid in the compartment. A catheter section associable with the syringe body includes a manually-secured anchor configured to maintain the catheter section in fluid communication with the pleural space of the subject following separation of the catheter section from the syringe body.

### CROSS-REFERENCE TO RELATED APPLICATIONS

The present application claims priority to and the benefit of U.S. patent application No. 63/500,130, “Visual and Tactile Verification and Securing System for Needle Thoracostomy (NT)” (filed May 4, 2023); and U.S. patent application No. 63/597,012, “Visual And Tactile Verification And Securing System For Needle Thoracostomy (NT)” (filed Nov. 8, 2023). All foregoing applications are incorporated herein by reference in their entireties for any and all purposes.

### TECHNICAL FIELD

The present disclosure relates to the field of medical devices.

### BACKGROUND

Existing devices for needle thoracostomy (NT) are difficult to use effectively and present a number of critical drawbacks to users and patients. Accordingly, there is a long-felt need in the art for improved NT devices.

### SUMMARY

In meeting the described long-felt needs, provided herein is a needle thoracostomy (NT) device, comprising: a syringe body, the syringe body including (i) a compartment having a fluid disposed therein, (ii) a plunger in fluid communication with the compartment, (iii) a resilient member biased against the plunger, and (iv) a needle in fluid communication with the compartment, the syringe body configured such that when the needle is advanced into a pleural space of a subject, the resilient member encourages movement of the plunger such that pleural air is communicated to the fluid disposed within the compartment of the syringe body and is visible as bubbles in the fluid; and a catheter section, the catheter section separably associable with the syringe body, and the catheter section including a manually-secured anchor configured to maintain the catheter section in fluid communication with the pleural space of the subject following separation of the catheter section from the syringe body.

Also provided is a NT device, comprising an indicator section, the indicator section comprising a variable-volume chamber having (i) a moveable boundary and (ii) a resilient member configured to encourage movement of the moveable boundary when the indicator section is in fluid communication with a pleural space of a subject such that pleural air is communicated to and visible in a fluid disposed within the chamber; and a catheter section separably associated with the indicator section, the catheter section comprising a manually-secured anchor configured to maintain the catheter section in fluid communication with the pleural space of the subject following separation of the catheter section from the indicator section.

Further provided is a method, comprising operating a NT device according to the present disclosure.

Additionally disclosed is a method, comprising: placing a lumen into fluid communication with a pleural space; effecting, with a resilient member, expansion of an enclosed volume in fluid communication with the lumen so as to encourage pleural air through the lumen into a fluid within the enclosed volume and effect formation of visible bubbles in the fluid; anchoring a catheter separably associated with the lumen so as to maintain the catheter in fluid communication with the pleural space of the subject; and separating the catheter from the lumen.

Further provided is a needle thoracostomy (NT) device, comprising: a body having a compartment, the compartment having a fluid disposed therein; a plunger; a catheter having a balloon associated therewith; a valve; and a needle, the device being configured such that when the needle is introduced into a pleural space of a subject and the plunger is operated to effect a negative pressure within the body, pleural air is transported to and visualized in the fluid disposed within the compartment of the body, the device being further configured such that when the plunger is operated to effect a positive pressure within the body, fluid is transported from the compartment of the body to the balloon through the valve, the valve operating to place the fluid into fluid communication with the balloon and to interrupt fluid communication between the fluid and pleural space, thereby securing the balloon within the pleural space.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various aspects discussed in the present document. In the drawings:

FIGS. 1A-1B illustrate an exemplary device in use. As shown in FIG. 1B, when the device has not entered the pleural space, no bubbles are seen within the device. As shown in FIG. 1B, a user can advance the device into the pleural space of a subject, and air is then drawn from the pleural space into the device and the air is then visualized as bubbles with the device, which bubbles indicate entry of the device into the pleural space. Also as shown in FIG. 1B, a valve can permit passage of the air into the device, where the air can be visualized as bubbles.

FIGS. 2A-2B illustrate the disclosed device in use. As shown in FIG. 2A, once the device is deployed into the pleural space, the user can exert a positive pressure on the fluid in the device, which positive pressure then inflates the balloon, thereby securing the balloon in place in the pleural space. As shown in FIG. 2A, the valve is closed, thereby preventing fluid from being exerted into the pleural space as the fluid is being exerted into the balloon. As shown in FIG. 2B, the syringe and needle can then be disengaged, leaving behind the catheter and the balloon, which are now secured in the pleural space and are available for use by medical personnel. FIG. 2C provides a depiction of an exemplary device according to the present disclosure.

FIG. 3 illustrates the need for the disclosed devices.

FIG. 4 describes the shortcomings of existing NT devices.

FIG. 5 illustrates a device according to the present disclosure. As shown, rather than manually applying negative pressure on the syringe to visualize bubbles in the chamber, a syringe can be spring loaded with a retention clip, which clip opposes motion of the plunger. The operator can first insert the needle into the chest wall and then remove the retention clip. The spring will then continually provide negative force, and when the needle arrives at an air filled pocket, air will be communicated through the needle and to the fluid within the syringe, and one can then visualize the bubbles in the chamber. This allows improved placement of the catheter without the need for increased manual dexterity, and also provides the novel feature of automatic negative pressure. One can select the spring such that the spring does not extend on its own or when the needle is not in fluid communication with an air pocket.

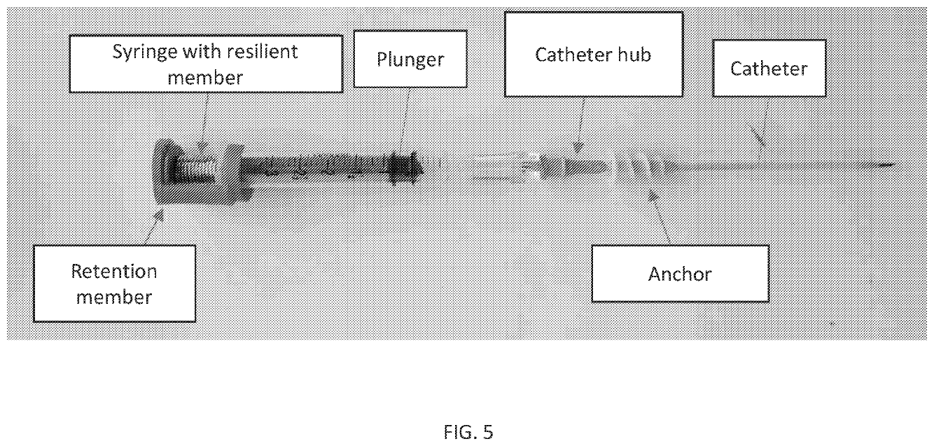

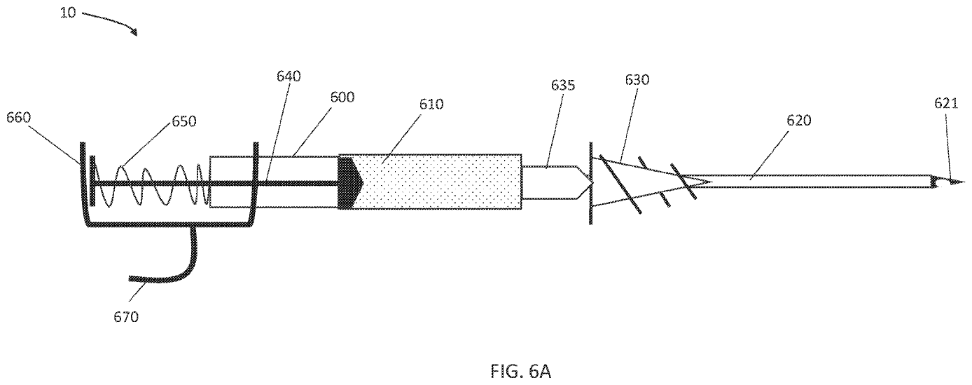

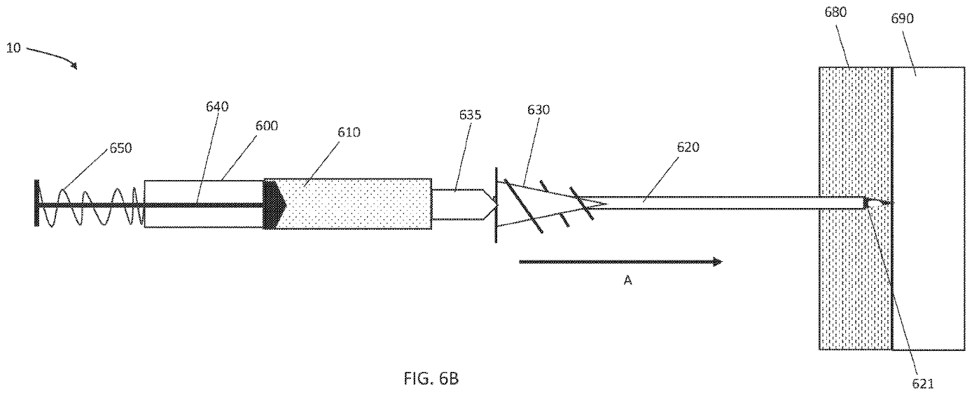

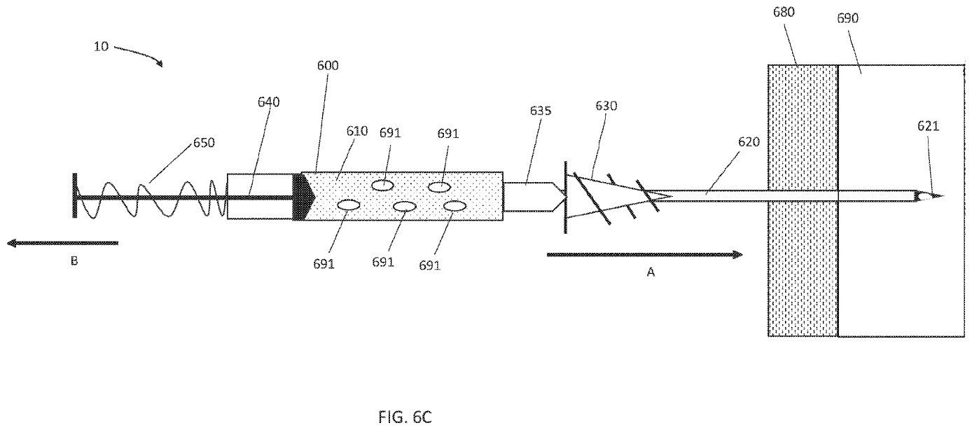

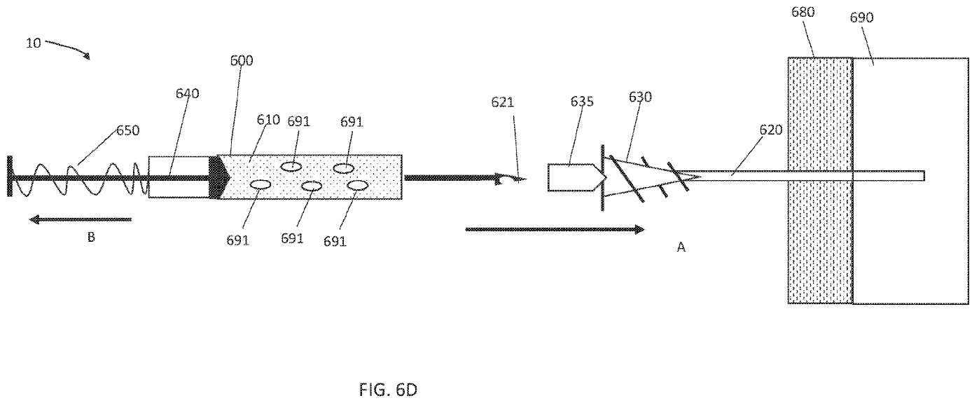

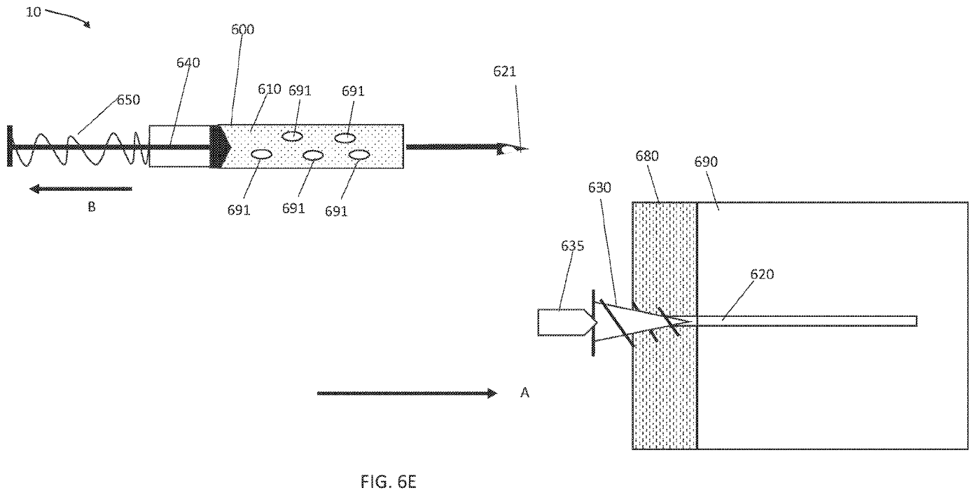

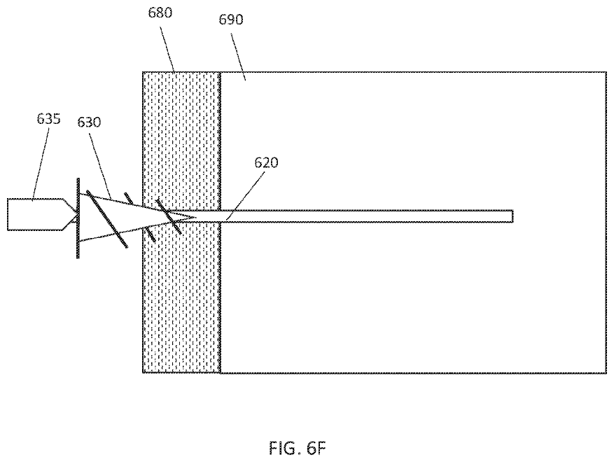

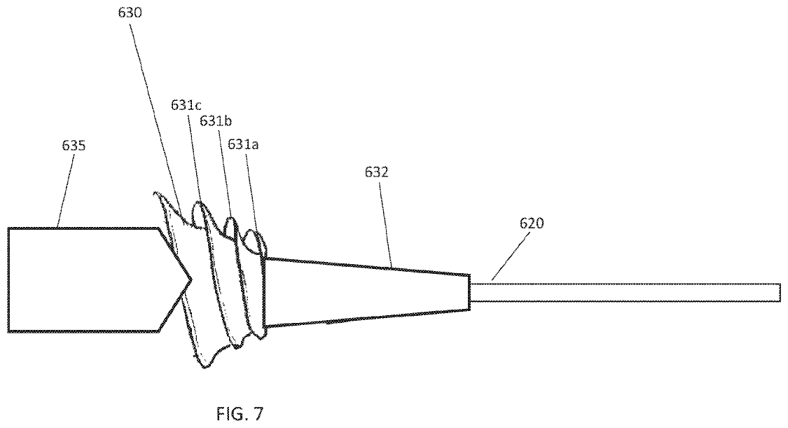

As shown in FIG. 5, a device according to the present disclosure can include an anchor. Such an anchor can be in the manner of a screw, such as a wood screw or a drywall screw-once an operator has visual verification that the needle has entered an air-filled pocket, the operator can advance the catheter and the screw is advanced into the skin so as to maintain the various components in position. The anchor can be configured such that the anchor is manually installable by pushing on the anchor so as to lodge the anchor into the subject's tissue, and the anchor will remain in place. To remove the anchor (which can be, for example, a threaded flange), one can twist the anchor like a typical screw. In this manner, installation can be achieved by pushing the anchor in, and the removal of the anchor can be achieved by twisting the anchor.

FIGS. 6A-6F depict an example device according to the present disclosure.

FIG. 7 depicts an example anchor and catheter section according to the present disclosure.

### DETAILED DESCRIPTION OF ILLUSTRATIVE EMBODIMENTS

The present disclosure may be understood more readily by reference to the following detailed description of desired embodiments and the examples included therein.

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing. All publications, patent applications, patents and other references mentioned herein are incorporated by reference in their entirety. The materials, methods, and examples disclosed herein are illustrative only and not intended to be limiting.

The singular forms “a,” “an,” and “the” include plural referents unless the context clearly dictates otherwise.

As used in the specification and in the claims, the term “comprising” can include the embodiments “consisting of” and “consisting essentially of.” The terms “comprise(s),” “include(s),” “having,” “has,” “can,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated ingredients/steps, which allows the presence of only the named ingredients/steps, along with any impurities that might result therefrom, and excludes other ingredients/steps.

As used herein, the terms “about” and “at or about” mean that the amount or value in question can be the value designated some other value approximately or about the same. It is generally understood, as used herein, that it is the nominal value indicated ±10% variation unless otherwise indicated or inferred. The term is intended to convey that similar values promote equivalent results or effects recited in the claims. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such. It is understood that where “about” is used before a quantitative value, the parameter also includes the specific quantitative value itself, unless specifically stated otherwise.

Unless indicated to the contrary, the numerical values should be understood to include numerical values which are the same when reduced to the same number of significant figures and numerical values which differ from the stated value by less than the experimental error of conventional measurement technique of the type described in the present application to determine the value.

All ranges disclosed herein are inclusive of the recited endpoint and independently of the endpoints. The endpoints of the ranges and any values disclosed herein are not limited to the precise range or value; they are sufficiently imprecise to include values approximating these ranges and/or values.

As used herein, approximating language can be applied to modify any quantitative representation that can vary without resulting in a change in the basic function to which it is related. Accordingly, a value modified by a term or terms, such as “about” and “substantially,” may not be limited to the precise value specified, in some cases. In at least some instances, the approximating language can correspond to the precision of an instrument for measuring the value. The modifier “about” should also be considered as disclosing the range defined by the absolute values of the two endpoints. For example, the expression “from about 2 to about 4” also discloses the range “from 2 to 4.” The term “about” can refer to plus or minus 10% of the indicated number. For example, “about 10%” can indicate a range of 9% to 11%, and “about 1” can mean from 0.9-1.1. Other meanings of “about” can be apparent from the context, such as rounding off, so, for example “about 1” can also mean from 0.5 to 1.4. Further, the term “comprising” should be understood as having its open-ended meaning of “including,” but the term also includes the closed meaning of the term “consisting.” For example, a composition that comprises components A and B can be a composition that includes A, B, and other components, but can also be a composition made of A and B only. Any documents cited herein are incorporated by reference in their entireties for any and all purposes.

Any embodiment or aspect provided herein is illustrative only and does not limit the scope of the present disclosure or the appended claims. Any part or parts of any one or more embodiments of aspects can be combined with any part or parts of any one or more other embodiments or aspects.

Tension pneumothorax is caused by air filling the space between the lungs and the chest wall. It causes death by obstructing blood return to the heart and by compromising ventilation and oxygenation. It occurs as a complication of traumatic injuries with an estimated prevalence between 3-20% of all trauma patients.

In the prehospital setting, the standard of care is to perform a life-saving and time-sensitive needle thoracostomy (NT). During this procedure, a needle is inserted into the pleural space through the skin to release accumulated air. This relieves the “tension” and serves as a bridge to definitive therapy. However, the success rate of NT in the field is highly variable, and ranges from 18-86%. In one study that examined post-placement CT scans, 94% of NT catheters failed to make pleural entry, and in another similar study, 76% of NT catheters were more than 5 mm away from making pleural entry, making this potentially life-saving procedure ineffective.

There are many potential reasons for failing to achieve pleural entry when performing NT, and difficulty verifying placement is a significant contributing factor.

Commercial devices for needle decompression require the operator to listen for a “rush of air” as the needle enters the pleural space to verify placement. Some more advanced (and more expensive) devices incorporate a whistle to make the sound louder or a spring-loaded needle that is safer to handle. However, in the prehospital setting, auditory verification is challenging and unreliable given noisy environments.

For this reason, provided here is a non-auditory verification system to confirm pleural entry. The disclosed technology has wide applicability in the commercial setting.

Current needle thoracostomy devices are frequently used by the military, to provide life-saving interventions in areas of conflict, and by prehospital personnel such as paramedics, to stabilize patients en route to the emergency department. Needle thoracostomy is also performed by physicians in the emergency department, trauma bay, ICU, and on rapid response teams to rapidly reverse this dangerous pathology.

The current standard of care includes utilizing a simple angiocatheter as a way to perform NT. But an angiocatheter is a suboptimal piece of equipment for needle thoracostomy, as it can easily become kinked, and has been documented to often fail achieving pleural access. However, it is a cheap and readily available alternative. As a way to circumvent these shortcomings, there are commercial NT kits. The standard NT kit is similar to an angiocatheter, but is redesigned ergonomically to aid in operator handling in the prehospital setting.

The disclosed technology is unique in multiple aspects. Whereas operators are currently required to rely on auditory cues to confirm entry, the disclosed device uses both visual and tactile cues to ensure placement. First, as pleural air mixes with the saline, the operator can easily visualize bubbles in the NT body. Second, tactile feedback is imparted as the operator attempts to inflate the catheter. It would be difficult to inflate the balloon on the catheter when it is in the subcutaneous tissue, and is only readily inflatable when it is in the pleural space. To date, the proposed device would be the only commercially available option to use visual and tactile confirmation to ensure that NT has been performed successfully.

An additional novel feature of the disclosed technology is the inflatable catheter, which secures the device in place and prevents dislodgement. NT catheters often become dislodged during long transport times because of patient movement or mishandling. By having our catheter inflated, it is tethered to the inner chest wall and resists any movement.

The disclosed device a number of unique features. First, there has never been an instance of an inflatable catheter that wraps around a needle. Similar devices, such as Foley catheters and Ward catheters are purely silicone in nature and correct placement of these catheters are predicated on other procedures (such as using a scalpel to penetrate a Bartholin's cyst) or using the lumen of a urethra to help guide a Foley. This technique of combining both access and stabilization into one device has important implications not only for NT, but potentially for other procedures as well.

Second, there is also no device on the market that allows for two disparate functions to occur while using a syringe. In our case, pulling back on the syringe will allow air to mix with the saline and verify the catheter location, and pushing on the syringe will translocate saline into the catheter. The idea of pulling back on a syringe plunger to achieve one effect, and then performing the opposite movement to achieve an entirely different effect has not been seen in medical devices previously.

Example components Penetration needle: This is the device component that allows penetration of superficial tissues and access to the pleural space. One design utilizes a 14 gauge, 4.0″ (10.2 cm) stainless steel needle which can be either a sharp or Veress tipped needle, depending on customer preference. This portion of the device is used to penetrate the superficial tissues (skin, subcutaneous tissue, intercostal muscles) at either the 2nd intercostal space in the midclavicular line or the 4-5th intercostal space in the anterior to midaxillary line. It is attached proximally to the NT Body (described below) via the entry channel. This needle is hollow bore to allow evacuation of air or blood through the entry channel and into the NT body.

NT body: The NT body can be a modified 5 or 10 cc translucent syringe with saline in the main compartment, an entry channel for pleural air or blood that is locked by a one-way valve, and a separate exit channel for saline to inflate the catheter (described below). As the penetrating needle is advanced through the skin, the operator draws on the syringe plunger to apply negative pressure within the syringe interior. Once the needle enters the pleural space, air will travel through the lumen of the penetrating needle into the NT body. This will also cause the operator to have a tactile sensation as air in the chamber will allow the easier application of negative pressure on the syringe. By passing through the one-way valve into the NT body, the pleural air will create bubbles in the saline. These bubbles in the main compartment will serve as visual confirmation that pleural entry has been achieved. Once verified, the operator can then push on the syringe to inject saline through the secondary exit channel and into the catheter with an integrated balloon. This balloon allows the operator to secure the device in place. The one-way valve mechanism will prevent the saline from exiting through the penetrating needle.

Catheter with integrated balloon: An example catheter is 3.125″ (8.25 cm) in length. It is made of PTFE coated silicone with a silicone medical balloon on the end. It fits snugly over the penetrating needle described above. The balloon can be inflated with sterile saline, serving 2 purposes. First, it secures the catheter in place inside the chest wall preventing dislodgement while transporting the patient. Second, it gives tactile feedback to the operator. If the operator tries to inflate the balloon too soon, they will feel resistance from the subcutaneous tissues resisting expansion of the balloon. When they are able to fill the balloon, they know that they have achieved pleural entry. After the balloon is inflated and the catheter is secured, the needle and NT body can be removed and safely disposed of. This is the end point of the device's deployment.

### Figures

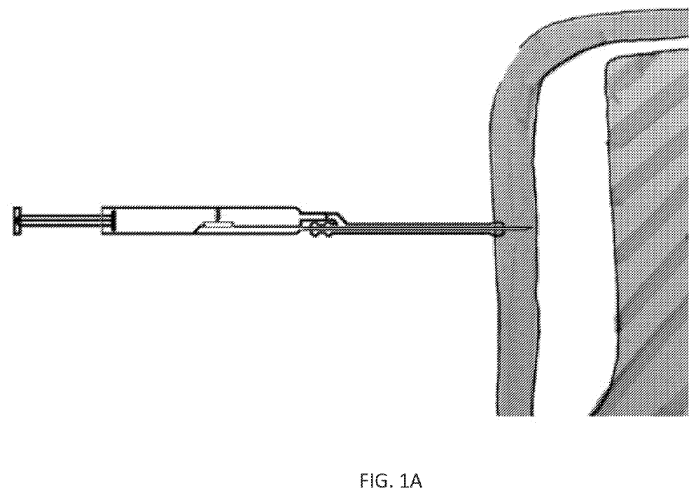

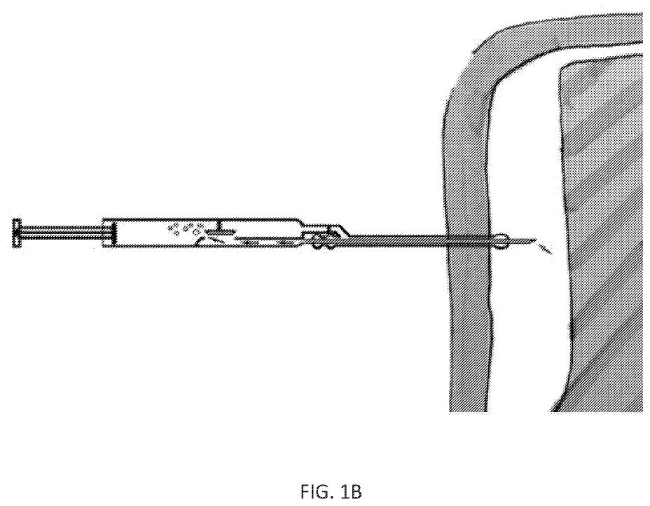

FIGS. 1A-1B illustrate an exemplary device in use. As shown in FIG. 1A, a user can introduce a device into the pleural space of a subject, and air is then drawn from the pleural space into the device and the air is then visualized as bubbles with the device, which bubbles indicate deployment of the device into the pleural space. Also as shown in FIG. 1A, a valve can permit passage of the air into the device, where the air can be visualized as bubbles. As shown in FIG. 1B, when the device has not entered the pleural space, no bubbles are seen.

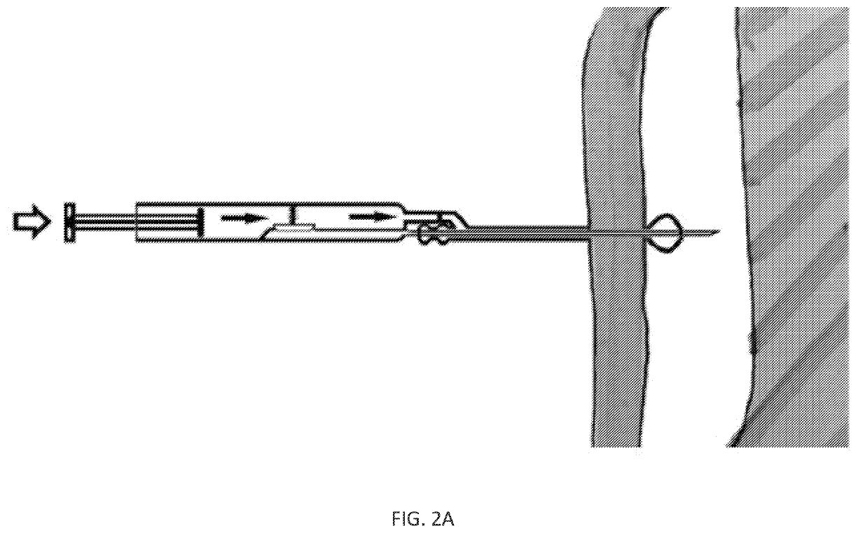

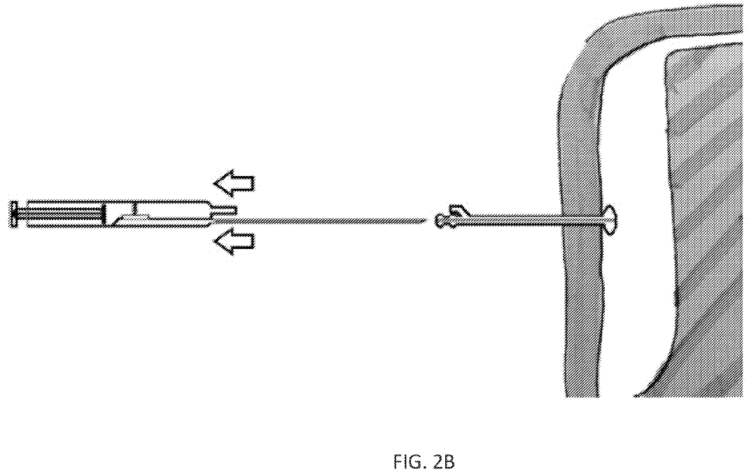

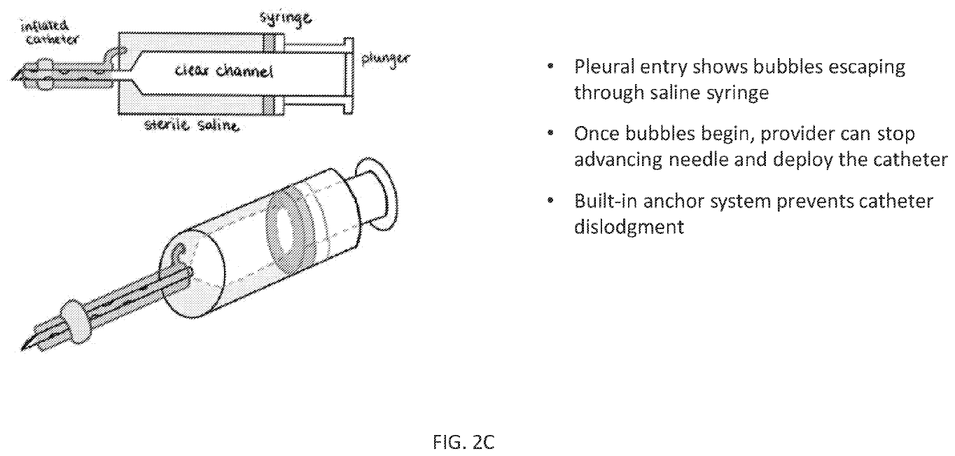

FIGS. 2A-2B illustrate the disclosed device in use. As shown in FIG. 2A,, once the device is deployed into the pleural space, the user can exert a positive pressure on the fluid in the device, which positive pressure then inflates the balloon, thereby securing the balloon in place in the pleural space. As shown in FIG. 2A, the valve is closed, thereby preventing fluid from being exerted into the pleural space as the fluid is being exerted into the balloon. As shown in FIG. 2B, the syringe and needle can then be disengaged, leaving behind the catheter and the balloon, which are now secured in the pleural space and are available for use by medical personnel. FIG. 2C provides a depiction of an exemplary device according to the present disclosure.

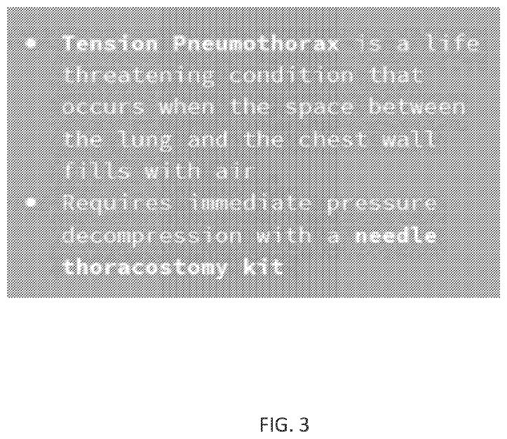

FIG. 3 illustrates the need for the disclosed devices.

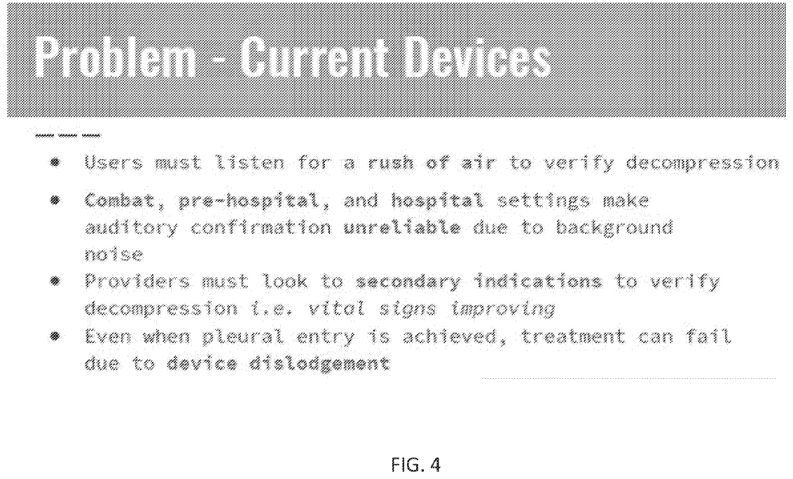

FIG. 4 describes the shortcomings of existing NT devices.

FIG. 5 illustrates a device according to the present disclosure. As shown, rather than manually applying negative pressure on the syringe to visualize bubbles in the chamber, a syringe can be spring loaded with a retention clip, which clip opposes motion of the plunger. The operator can first insert the needle into the chest wall and then remove the retention clip. The spring will then continually provide negative force, and when the needle arrives at an air filled pocket, air will be communicated through the needle and to the fluid within the syringe, and one can then visualize the bubbles in the chamber. This allows improved placement of the catheter without the need for increased manual dexterity, and also provides the novel feature of automatic negative pressure. One can select the spring such that the spring does not extend on its own or when the needle is not in fluid communication with an air pocket.

As shown in FIG. 5, a device according to the present disclosure can include an anchor. Such an anchor can be in the manner of a screw, such as a wood screw or a drywall screw-once an operator has visual verification that the needle has entered an air-filled pocket, the operator can advance the catheter and the screw is advanced into the skin so as to maintain the various components in position. The anchor can be configured such that the anchor is manually installable by pushing on the anchor so as to lodge the anchor into the subject's tissue, and the anchor will remain in place. To remove the anchor (which can be, for example, a threaded flange), one can twist the anchor like a typical screw. In this manner, installation can be achieved by pushing the anchor in, and the removal of the anchor can be achieved by twisting the anchor.

FIGS. 6A-6F depict an example device according to the present disclosure.

As shown in FIG. 6A, a device 10 can include syringe body 600. Fluid 610 can reside in a compartment within syringe body 600; as shown, the compartment can be defined within syringe body 600. Plunger 640 can define the compartment in which fluid 610 is disposed.

Resilient member 650 can be biased against plunder 640. Although resilient member 650 in FIG. 6A is a spring, this is not a requirement, as resilient member 650 need not be a spring. As one example, resilient member 650 can also be, for example, an elastomer.

NT device 10 can also include catheter section 620. Needle 621 can be present at the end of catheter section 620. Catheter section 620 can be separably associable with syringe body 600. As shown, catheter section 620 can include anchor 630. Anchor 630 can be configured to maintain the catheter section in fluid communication with the pleural space of a subject following separation of the catheter section from the syringe body. Anchor 630 can be configured such that the anchor is manually installable by pushing on the anchor so as to lodge the anchor into the subject's tissue, and the anchor will remain in place. To remove the anchor (which can be, for example, a threaded flange), one can twist the anchor like a typical screw. In this manner, installation can be achieved by pushing the anchor in, and the removal of the anchor can be achieved by twisting the anchor. Anchor 630 can, for example, have the shape of a shark egg or other threaded configuration that allows for insertion via pushing to lodge the anchor into the subject's tissue, and is then removed by twisting like a typical screw. Device 10 can also include catheter hub 635. Catheter hub 635 can be slidable along catheter section 620, but this is not a requirement. In some embodiments, anchor 620 includes a catheter hub.

Also as shown in FIG. 6A, device 10 can include retention member 660. Retention member 660 can be, for example, a clip. Retention member 660 can include element 670, which element can be a hook, loop, opening, ridge, slot, groove, or other element that facilitates the retention member's removal. A retention member can be configured so that the retention member is removable with one hand. Device 10 can be configured such that, with one hand, the device can be inserted and the retention member removed. The device can even be configured such that, with one hand, the user can install the anchor into the subject. In this way, a user can deploy the device using only one hand.

FIG. 6B shows the insertion of a device 10 in direction A into the skin 680 of a subject, with the user's pleural space 690 lying behind skin 680. Retention member 660 (now not shown) has been removed from the device. Because needle 621 has not yet entered the pleural space of the subject, resilient member 650 cannot not move plunger 640 outward.

In FIG. 6C, needle 621 has entered pleural space 690. Resilient member 650 can then encourage plunger 640 outward—in direction B—which in turn draws air from pleural space 690 into fluid 610. The air is visible as bubbles 691 in fluid 610, and the presence of bubbles 691 indicates to the user that the catheter has entered the pleural space 690 of the subject.

Although the bubbles can be visualized by a user, a device according to the present disclosure can be constructed so as to provide the user with a signal-which can be audible or tactile-when bubbles are detected. For example, a device can provide a beep or chirp sound to the user. Alternatively, a device can vibrate or provide other haptic feedback when bubbles are detected. In this way, a user can receive any one or more of aural, visual, and haptic feedback when bubbles are detected.

As shown in FIG. 6D, syringe 600 and needle 621 have been withdrawn from catheter section 620, the end of which is within pleural space 690. As shown, anchor 630 and catheter 635 remain at a distance from skin 620.

As shown in FIG. 6E, the anchor 630 can be installed into tissue 680. In some embodiments, anchor 630 is slidably associated with catheter section 620. This is not a requirement, however, as the user can continue advancing catheter section 620 into pleural space 690 until anchor 630 is installed; this can be facilitated when catheter section 630 is flexible. In some embodiments, catheter hub 635 is separate from anchor 630; in some embodiments, catheter hub 635 is connected to anchor 630. In still other embodiments, anchor 630 comprises a catheter hub; as but one example, anchor 630 can be a flanged catheter hub.

FIG. 6F shows catheter section 620 in pleural space 690 and anchor section 630 installed in tissue 680; syringe 600 and needle 621 have been disengaged.

FIG. 7 provides an example anchor 630, as shown relative to catheter section 620 and catheter hub 635. As shown, anchor 630 can include distal end 632, which can be tapered or even pointed to facilitate insertion into a subject. Anchor 630 can also include threaded flanges; such flanges can be arranged so that anchor 630 can be placed securely by insertion and can be removed by unscrewing.

As shown, anchor 630 can include helical threads (631a, 631b, and 631c) that increase in diameter along the length of anchor 630. It should be understood, however, that the configuration shown in FIG. 7 is exemplary only and is not limiting. As shown, catheter hub 635 can be connected to anchor 630. This is, however, not a requirement, as anchor 630 can itself be configured to be a catheter hub.

### Aspects

The following Aspects are illustrative only and do not limit the scope of the present disclosure or the appended claims. Any part or parts of any one or more Aspects can be combined with any part or parts of any one or more other Aspects.

Aspect 1. A needle thoracostomy (NT) device, comprising: a syringe body, the syringe body including (i) a compartment having a fluid disposed therein, (ii) a plunger in fluid communication with the compartment, (iii) a resilient member biased against the plunger, and (iv) a needle in fluid communication with the compartment, the syringe body configured such that when the needle is advanced into a pleural space of a subject, the resilient member encourages movement of the plunger such that pleural air is communicated to the fluid disposed within the compartment of the syringe body and is visible as bubbles in the fluid; and a catheter section, the catheter section separably associable with the syringe body, and the catheter section including a manually-secured anchor configured to maintain the catheter section in fluid communication with the pleural space of the subject following separation of the catheter section from the syringe body.

Aspect 2. The NT device of Aspect 1, further comprising a retention member, the retention member configured to restrict movement of the plunger in response to the resilient member, the retention member optionally comprising at least one of a spring and an elastomer.

Aspect 3. The NT device of Aspect 2, wherein the retention member is removable from the NT device.

Aspect 4. The NT device of any one of Aspects 2-3, wherein the retention member is characterized as a clip, the clip optionally being removable with one hand. The retention member can include, for example, a tab, grip, flange, hook, or other element to facilitate the retention member's removal. Without being bound to any particular theory, the retention member can include a hook that a user can engage with a finger, thumb, or both to then remove the retention members from the NT device.

Aspect 5. The NT device of any one of Aspects 1-4, wherein the anchor comprises a threaded flange.

Aspect 6. The NT device of Aspect 5, wherein the threaded flange is arranged such that the anchor can be anchored in a medium by pushing and removable from the medium by unscrewing.

Aspect 7. The NT device of any one of Aspects 1-6, wherein the anchor is slidably associated with the catheter section.

Aspect 8. The NT device of any one of Aspects 1-7, wherein the anchor comprises a catheter hub.

Aspect 9. The NT device of any one of Aspects 1-7, further comprising a catheter hub.

Aspect 10. The NT device of Aspect 9, wherein the catheter hub is secured to the anchor.

Aspect 11. The NT device of Aspect 10, wherein the catheter hub is slidably associated with the catheter section.

Aspect 12. The NT device of any one of Aspects 1-11, wherein the resilient member comprises at least one of a spring and an elastomer.

Aspect 13. A NT device, comprising: an indicator section, the indicator section comprising a variable-volume chamber having (i) a moveable boundary and (ii) a resilient member configured to encourage movement of the moveable boundary when the indicator section is in fluid communication with a pleural space of a subject such that pleural air is communicated to and visible in a fluid disposed within the chamber; and a catheter section separably associated with the indicator section, the catheter section comprising a manually-secured anchor configured to maintain the catheter section in fluid communication with the pleural space of the subject following separation of the catheter section from the indicator section.

Aspect 14. The NT device of Aspect 13, wherein the indicator section is characterized as a syringe.

Aspect 15. The NT device of Aspect 14, wherein the variable-volume chamber is an internal chamber of the syringe, the internal chamber being bounded by a moveable plunger.

Aspect 16. The NT device of Aspect 15, wherein the resilient member encourages movement of the moveable plunger.

Aspect 17. The NT device of any one of Aspects 13-16, wherein the fluid is saline.

Aspect 18. The NT device of any one of Aspects 13-17, further comprising a retention member, the retention member configured to restrict movement of the moveable boundary of the variable-volume chamber.

Aspect 19. The NT device of any one of Aspects 13-18, wherein the resilient member comprises at least one of a spring and an elastomer.

Aspect 20. A method, comprising operating a NT device according to any one of Aspects 1-19

Aspect 21. A method, comprising: placing a lumen into fluid communication with a pleural space; effecting, with a resilient member, expansion of an enclosed volume in fluid communication with the lumen so as to encourage pleural air through the lumen into a fluid within the enclosed volume and effect formation of visible bubbles in the fluid; anchoring a catheter separably associated with the lumen so as to maintain the catheter in fluid communication with the pleural space of the subject; and separating the catheter from the lumen.

Aspect 22. A needle thoracostomy (NT) device, comprising: a body having a compartment, the compartment having a fluid disposed therein; a plunger; a catheter having a balloon associated therewith; a valve; and a needle, the device being configured such that when the needle is introduced into a pleural space of a subject and the plunger is operated to effect a negative pressure within the body, pleural air is transported to and visualized in the fluid disposed within the compartment of the body, the device being further configured such that when the plunger is operated to effect a positive pressure within the body, fluid is transported from the compartment of the body to the balloon through the valve, the valve operating to place the fluid into fluid communication with the balloon and to interrupt fluid communication between the fluid and pleural space, thereby securing the balloon within the pleural space.

Aspect 23. The NT device of Aspect 22, wherein the catheter is separable from the body.

Aspect 24. The NT device of any one of Aspects 22-23, wherein the needle is slidably engageable with the catheter.

Aspect 25. A NT device comprising a needle, the NT device being configured to provide a visual indication of deployment of the needle into the pleural space of the subject.

Aspect 26. The NT device of Aspect 25, wherein the visual indication comprises bubbling.

Aspect 27. A method, comprising the use of a NT device according to any one of Aspects 22-26.

1. A needle thoracostomy (NT) device, comprising:
a syringe body,
the syringe body including (i) a compartment having a fluid disposed therein, (ii) a plunger in fluid communication with the compartment, (iii) a resilient member biased against the plunger, and (iv) a needle in fluid communication with the compartment,
the syringe body configured such that when the needle is advanced into a pleural space of a subject, the resilient member encourages movement of the plunger such that pleural air is communicated to the fluid disposed within the compartment of the syringe body and is visible as bubbles in the fluid; and
a catheter section,
the catheter section separably associable with the syringe body, and
the catheter section including a manually-secured anchor configured to maintain the catheter section in fluid communication with the pleural space of the subject following separation of the catheter section from the syringe body.

2. The NT device of claim 1, further comprising a retention member, the retention member configured to restrict movement of the plunger in response to the resilient member, the retention member optionally comprising at least one of a spring and an elastomer, and the retention member optionally being removable from the NT device.

3. The NT device of claim 2, wherein the retention member is removable from the NT device.

4. The NT device of claim 2, wherein the retention member is characterized as a clip, the clip optionally being removable with one hand.

5. The NT device of claim 1, wherein the anchor comprises a threaded flange.

6. The NT device of claim 5, wherein the threaded flange is arranged such that the anchor can be anchored in a medium by pushing and removable from the medium by unscrewing.

7. The NT device of claim 1, wherein the anchor is slidably associated with the catheter section.

8. The NT device of claim 1, wherein the anchor comprises a catheter hub.

9. The NT device of claim 1, further comprising a catheter hub.

10. The NT device of claim 9, wherein the catheter hub is secured to the anchor, the catheter hub optionally slidably associated with the catheter section.

11. (canceled)

12. (canceled)

13. A NT device, comprising:
an indicator section,
the indicator section comprising a variable-volume chamber having (i) a moveable boundary and (ii) a resilient member configured to encourage movement of the moveable boundary when the indicator section is in fluid communication with a pleural space of a subject such that pleural air is communicated to and visible in a fluid disposed within the chamber; and
a catheter section separably associated with the indicator section,
the catheter section comprising a manually-secured anchor configured to maintain the catheter section in fluid communication with the pleural space of the subject following separation of the catheter section from the indicator section.

14. The NT device of claim 13, wherein the indicator section is characterized as a syringe, and optionally wherein the variable-volume chamber is an internal chamber of the syringe, the internal chamber being bounded by a moveable plunger.

15. (canceled)

16. The NT device of claim 15, wherein the variable-volume chamber is an internal chamber of the syringe, the internal chamber being bounded by a moveable plunger, and wherein the resilient member encourages movement of the moveable plunger.

17. (canceled)

18. The NT device of claim 13, further comprising a retention member, the retention member configured to restrict movement of the moveable boundary of the variable-volume chamber.

19. (canceled)

20. (canceled)

21. A method, comprising:
placing a lumen into fluid communication with a pleural space of a subject;
effecting, with a resilient member, expansion of an enclosed volume in fluid communication with the lumen so as to encourage pleural air through the lumen into a fluid within the enclosed volume and effect formation of visible bubbles in the fluid;
anchoring a catheter separably associated with the lumen so as to maintain the catheter in fluid communication with the pleural space of the subject; and
separating the catheter from the lumen.

22. A needle thoracostomy (NT) device, comprising:
a body having a compartment, the compartment having a fluid disposed therein;
a plunger;
a catheter having a balloon associated therewith;
a valve;
and
a needle,
the device being configured such that when the needle is introduced into a pleural space of a subject and the plunger is operated to effect a negative pressure within the body, pleural air is transported to and visualized in the fluid disposed within the compartment of the body,
the device being further configured such that when the plunger is operated to effect a positive pressure within the body, fluid is transported from the compartment of the body to the balloon through the valve, the valve operating to place the fluid into fluid communication with the balloon and to interrupt fluid communication between the fluid and pleural space, thereby securing the balloon within the pleural space.

23. The NT device of claim 22, wherein the catheter is separable from the body.

24. The NT device of claim 22, wherein the needle is slidably engageable with the catheter.

25. A NT device comprising a needle, the NT device being configured to provide a visual indication of deployment of the needle into a pleural space of a subject.

26. The NT device of claim 25, wherein the visual indication comprises bubbling.

27. (canceled)
